(19) United States
(12) Patent Application Publication
(10) Pub. No.: US 2024/0208888 A1
(43) Pub. Date: Jun. 27, 2024

(54) PROCESS AND PLANT FOR PRODUCING METHANOL FROM SUBSTOICHIOMETRIC SYNTHESIS GAS

(71) Applicant: L'Air Liquide, Société Anonyme pour I'Etude et I'Exploitation des Procédés Georges Claude, Paris (FR)

(72) Inventors: Veronika GRONEMANN, Karben (DE); Michael WILKEN, Frankfurt am Main (DE); Tobias OELMANN, Bad Vilbel (DE)

(21) Appl. No.: 18/288,310

(22) PCT Filed: Apr. 26, 2022
(86) PCT No.: PCT/EP2022/060999

§ 371 Date: Oct. 25, 2023

(30) Foreign Application Priority Data
Apr. 30, 2021 (EP) 21020241.2

(51) Int. Cl. C07C 29/151; C01B 3/32

(52) U.S. Cl. CPC C07C 29/1518; C01B 3/32; C01B 2203/061; C01B 2210/001; C01B 2210/0014

(57) ABSTRACT

The invention relates to a process and to a plant for producing methanol from a synthesis gas having a hydrogen deficit. A fresh gas stream from a reformer unit which includes hydrogen and carbon oxides is combined with a hydrogen-containing stream from a hydrogen recovery stage. This affords a synthesis gas stream having a stoichiometry number SN, defined as SN=[n(H₂)−n(CO₂)]/[n(CO)+n(CO₂)], of less than 2.0. The synthesis gas stream is combined with a residual gas stream and the synthesis gas stream and the residual gas stream are passed through a bed of a methanol synthesis catalyst at elevated pressure and elevated temperature to obtain a product stream having methanol and the residual gas stream, and the product stream is cooled to separate methanol from the residual gas stream.

### CROSS REFERENCE TO RELATED APPLICATIONS

This application is a 371 of International Application No. PCT/EP2022/060999, filed Apr. 26, 2022, which claims priority to European Patent Application No. 21020241.2, filed Apr. 30, 2021, the entire contents of which are incorporated herein by reference.

### BACKGROUND

### Field of the Invention

The invention relates to a process and a plant for producing methanol from a make-up gas stream, wherein the make-up gas stream is admixed with a hydrogen-containing stream to obtain a synthesis gas stream having a stoichiometry number of less than 2.0. The invention further relates to the use of the process according to the invention or of the plant according to the invention for producing methanol from make-up gas produced by autothermal reforming and/or partial oxidation and/or combined reforming and/or pyrolysis of biomass.

### Prior Art

On a large industrial scale methanol is produced from synthesis gas. Synthesis gas is a mixture of predominantly hydrogen (H₂), carbon monoxide (CO) and carbon dioxide (CO₂). It further comprises smaller amounts of gas constituents inert under the conditions of methanol synthesis. Carbon monoxide and carbon dioxide are often subsumed in the term “carbon oxide”. In the process today described as low-pressure methanol synthesis the synthesis gas is converted into methanol and water (as a necessarily generated by-product) at a synthesis pressure of 60 to 120 bar. After compression to the respective synthesis pressure the employed synthesis gas, often referred to as make-up gas, is passed through a catalyst bed of a methanol synthesis catalyst at catalyst temperatures of typically more than 200° C. The methanol synthesis catalyst is typically a composition comprising copper as the catalytically active species. Depending on the process mode one or more serially arranged or parallel reactors, each having an appropriate catalyst bed, are employed. The conversion of the carbon oxides into methanol and water over the catalyst is incomplete on account of the establishment of a thermodynamic equilibrium according to the reactions

$${CO}_{2} + 3 {H}_{2} \vec{↽} {CH}_{3} OH + {H}_{2} O ,$$

$$CO + 2 {H}_{2} \vec{↽} {CH}_{3} OH .$$

As a result, the production process is typically run as a recirculating process in a so-called synthesis loop. The reaction mixture obtained at the reactor outlet is cooled to below the boiling point of methanol to separate methanol and water from the circuit. Simultaneously, the unconverted synthesis gas, often referred to as recycle gas or residual gas, is mixed with make-up gas and recycled to the methanol synthesis catalyst for renewed reaction. A substream of unconverted synthesis gas is continuously withdrawn as a purge gas stream to avoid the concentration of inert constituents in the synthesis loop increasing over time.

The composition of the make-up gas or of a synthesis gas is generally characterized by the so-called stoichiometry number SN defined as

$$SN = \frac{n ( {H}_{2} ) - n ( {CO}_{2} )}{n ( CO ) + n ( {CO}_{2} )} , with n in [ mol ] .$$

A make-up gas composition stoichiometrically balanced for methanol synthesis is characterized by a stoichiometry number SN of 2.0. Values of less than 2.0 indicates a hydrogen deficit, while values of greater than 2.0 indicate a hydrogen excess.

Synthesis gases having a hydrogen deficit are obtained for example in processes comprising a partial oxidation step or in the production of synthesis gas by coal gasification. In such a case the hydrogen is virtually completely consumed in the methanol synthesis, while a substantial portion of the carbon oxides is not converted. This results in a composition in the synthesis loop which features high proportions of carbon oxides but a low proportion of hydrogen. This inter alia has the result that the content of by-products (in particular higher alcohols and ketones) is higher than desired and that the maximum achievable methanol yield is reduced.

In order also to allow advantageous use of hydrogen-poor make-up gas having having a stoichiometry number of markedly below 2.0 in methanol production, the synthesis gas may be adjusted to a higher stoichiometry number using hydrogen from a hydrogen recovery plant for example. This is possible for example through hydrogen recovery from the purge stream.

EP 3 205 622 B1 discloses a process wherein unconverted synthesis gas referred to as residual gas is sent partially (as purge gas) to a hydrogen recovery stage. This affords a hydrogen-containing stream which is admixed with the make-up gas stream. The resulting mixture is subsequently compressed to synthesis pressure and converted into methanol.

However, the hydrogen amounts obtainable from the substream of the unconverted synthesis gas are often insufficient to obtain a synthesis gas having an adequately high stoichiometry number. For example synthesis gases having a high hydrogen deficit may require such a high purge stream proportion for hydrogen recovery that the synthesis loop must either be operated at low pressures or that the ratio of the recycle gas stream to the make-up gas stream must be set low.

To counter these disadvantages it is also conceivable to divert a portion of the make-up gas upstream of the methanol synthesis and send it to a hydrogen recovery stage. The disadvantage of this arrangement is that in the hydrogen recovery stage a portion of the hydrogen is lost before it passes into the synthesis circuit. Furthermore, after enrichment with this hydrogen, the synthesis gas has such a high stoichiometry number that a purge gas stream not utilized in this case may comprise a considerable portion of unconverted hydrogen.

U.S. Pat. No. 7,786,180 B2 therefore proposes supplying the hydrogen recovery stage with a mixed stream of make-up gas and purge gas to at least partially overcome the abovementioned disadvantages. The disadvantage of this arrangement is that the make-up gas stream must be throttled by a pressure reduction valve in order at least to equalize the pressure drop generated by the hydrogen recovery stage. The pressure thus lost in the make-up gas conduit must be compensated in the subsequent compression to synthesis pressure.

Post-published European patent application EP 19020610.2 therefore discloses a process and a plant for producing methanol from a synthesis gas having a hydrogen deficit, wherein a make-up gas stream from a reformer unit comprising hydrogen and carbon oxides is admixed with a hydrogen-containing stream from a hydrogen-recovery stage. This affords a hydrogen-rich synthesis gas stream having a stoichiometry number SN, defined as SN=[n(H₂)−n(CO₂)]/[n(CO)+n(CO₂)], of not less than 2.0. The hydrogen-rich synthesis gas stream is combined with a residual gas stream and the hydrogen-rich synthesis gas stream and the residual gas stream are passed through a bed or two or more beds of a methanol synthesis catalyst at elevated pressure and elevated temperature to obtain a product stream comprising methanol and the residual gas stream, and the product stream is cooled to separate methanol from the residual gas stream. It is provided that a portion of the residual gas stream is separated as a purge gas stream and a portion of the hydrogen-rich synthesis gas stream is separated and combined with the purge gas stream to obtain a mixed synthesis gas stream and the mixed synthesis gas stream is sent to the hydrogen recovery stage for producing the hydrogen-containing stream.

In contrast to U.S. Pat. No. 7,786,180 B2, this does not comprise sending the make-up gas stream and the purge gas stream diverted from the residual gas stream to the hydrogen recovery stage but, on the contrary, comprises sending the hydrogen-rich synthesis gas stream already adjusted with hydrogen to a stoichiometry number of not less than 2.0 to the hydrogen recovery stage together with the purge gas stream. This makes it possible to eschew a throttling of the make-up gas stream to divert a portion of the make-up gas stream in the direction of the hydrogen recovery stage. Savings in relation to the compression energy to be expended are further achieved.

Synthesis gases supplied to the methanol reactor characterized by a stoichiometry number SN of 2.0 or more can result in an excess of hydrogen in the circuit of the methanol synthesis which increases over time and thus necessitates increasing the dimensions of the components in the methanol synthesis circuit.

Synthesis gases characterized by an SN of 2.0 or more further do not fully exploit the potential of modern methanol synthesis catalysts which achieve sufficient total carbon conversions over the catalyst bed even in the case of synthesis gases having a low stoichiometry number.

It has further been found that the adjustment of a high stoichiometry number SN of 2.0 or more according to the process of EP 19020610.2 results in an elevated formation of methanol from carbon dioxide and is additionally associated with an elevated overall consumption of hydrocarbon-containing input, in particular natural gas, having regard to the reformer unit arranged upstream of the methanol synthesis. However, it is sought to minimize the consumption of the input to the reforming unit arranged upstream of the methanol synthesis plant as far as possible. The formation of methanol from carbon dioxide is also to be minimized as far as possible since the increased formation of methanol from carbon dioxide is associated with an increased formation of water which requires separation in a downstream workup step.

Furthermore, high carbon dioxide conversions have the result that altogether less purge gas is generated or producible despite being required for producing the hydrogen-containing stream in the hydrogen recovery stage. If only a small amount of purge gas is available for production of the hydrogen-containing stream, the hydrogen for adjusting the stoichiometry number of the synthesis gas must be obtained to a greater extent from reformed gas, which is undesirable. In this case a greater portion of the hydrogen is lost in the hydrogen recovery stage, even before it passes into the synthesis circuit for the methanol synthesis.

### SUMMARY

It is an object of the present invention to provide a process and a plant for producing methanol which at least partially overcomes the disadvantages of the prior art.

It is a further object of the present invention to minimize the consumption of hydrocarbon-containing inputs, in particular of natural gas, of a reformer unit arranged upstream of the methanol synthesis.

It is a further object of the present invention to minimize the formation of methanol from carbon dioxide in the synthesis loop in the methanol synthesis.

The independent claims make a contribution to the at least partial achievement of at least one of the above objects. The dependent claims provide preferred embodiments which contribute to the at least partial achievement of at least one of the objects. Preferred embodiments of constituents of one category according to the invention are, where relevant, likewise preferred for identically named or corresponding constituents of a respective other category according to the invention.

The terms “having”, “comprising” or “containing”, etc., do not preclude the possible presence of further elements, ingredients, etc. The indefinite article “a” does not preclude the possible presence of a plurality.

The abovementioned objects are at least partially achieved by a process for producing methanol, wherein a make-up gas stream from a reformer unit comprising hydrogen and carbon oxides is admixed with a hydrogen-containing stream from a hydrogen recovery stage to obtain a synthesis gas stream having a stoichiometry number SN, defined as SN=[n(H₂)−n(CO₂)]/[n(CO)+n(CO₂)], of less than 2.0, and wherein the synthesis gas stream is combined with a residual gas stream and the synthesis gas stream and the residual gas stream are passed through a bed of a methanol synthesis catalyst at elevated pressure and elevated temperature to obtain a product stream comprising methanol and the residual gas stream, and wherein the product stream is cooled to separate methanol from the residual gas stream, and wherein a portion of the residual gas stream is separated as a purge gas stream and sent to the hydrogen recovery stage for producing the hydrogen-containing stream.

In one embodiment of the process the synthesis gas stream and the residual gas stream are passed through a plurality of beds of a methanol synthesis catalyst at elevated pressure and elevated temperature.

In one embodiment the plurality of beds of the methanol synthesis catalyst is arranged in series.

In one embodiment the plurality of beds of the methanol synthesis catalyst is arranged in parallel.

The synthesis gas supplied to the bed or the beds of the methanol synthesis catalyst together with the residual gas stream has a stoichiometry number SN of less than 2.0, i.e. is substoichiometric having regard to the production of methanol and the hydrogen proportion required therefor. It has been found that especially in conjunction with modern methanol synthesis catalysts the use of substoichiometric synthesis gas in the context of the process according to the invention made it possible to at least partially overcome the abovementioned disadvantages. It has further been found that in conjunction with the definition of further parameters such as
the stoichiometry number at the inlet to the catalyst bed which results from the synthesis gas stream and the residual gas stream,
the profile of the catalyst bed temperature and
the operating temperature of the methanol reactor, substoichiometric synthesis gas too may be used to obtain a crude methanol product which allows production of methanol conforming to specification with standard workup methods, in particular distillation/rectification.

The process according to the invention further allows the use of hydrogen-poor, substoichiometric make-up gas streams, featuring a stoichiometry number of markedly below 2.0.

It has further been found that the use of substoichiometric synthesis gas reduces the formation of methanol from carbon dioxide, thus reducing the carbon dioxide-containing input consumption of the upstream reformer unit.

The reduced formation of methanol from carbon dioxide, i.e. the reduced conversion of carbon dioxide in the synthesis loop, has the result that sufficient purge gas may be withdrawn from the residual gas stream to preclude a portion of the synthesis gas stream needing to be diverted and supplied to the hydrogen recovery stage together with the purge gas stream to generate sufficient hydrogen in the hydrogen recovery stage for adjustment of the stoichiometry number of the synthesis gas.

The lower conversions of carbon dioxide in the synthesis loop further result in the formation of less water requiring later removal in the context of the workup of the crude product.

The make-up gas stream is preferably a synthesis gas stream from a reformer unit which especially features a deficit of hydrogen and where the stoichiometry number of the make-up gas is less than 2.0, in particular less than 1.90, or less than 1.80, or less than 1.70, or less than 1.60. Such a make-up gas stream is especially produced in a reformer unit which comprises a partial oxidation step of a carbon-containing input gas to produce the synthesis gas. For example, the make-up gas stream may be produced from autothermal reforming of a carbon-containing input gas. The input gas is preferably natural gas. The make-up gas stream may further be produced from coal gasification. Prior to the admixing of the hydrogen-containing stream and compression to synthesis pressure, the make-up gas stream is cooled to a temperature of preferably not more than 40° C. for condensation and separation of water. The make-up gas stream typically has a pressure between 20 and 60 bar, which is why an additional compression to synthesis pressure is required prior to the conversion over the methanol synthesis catalyst.

The reformer unit is a unit known to those skilled in the art for conversion (reforming) of a gaseous, liquid or solid hydrocarbon-containing input stream into synthesis gas. Examples of reformer units known to those skilled in the art include steam reformers (SMR), reformers for partial oxidation of gases or liquids, autothermal reformers (ATR), combinations thereof such as for example combined reformers (combination of SMR and ATR), coal gasifiers and gasifiers for biomass. One example of a gaseous hydrocarbon-containing input material is natural gas. The main component of natural gas is methane. Examples of solid carbon-containing input materials are coal, solid wastes (refuse) and biomass. The reformer unit is especially a reformer unit providing substoichiometric synthesis gas having a stoichiometry number of less than 2.0.

In a preferred embodiment of the invention the make-up gas stream is provided from a reformer unit which converts the input natural gas into synthesis gas.

The hydrogen-containing stream preferably has a hydrogen content of not less than 80% by volume. In one embodiment the hydrogen-containing stream has a hydrogen content of not less than 85% by volume, or of not less than 90% by volume, or of not less than 95% by volume, or of not less than 99% by volume. A hydrogen-containing stream containing pure or substantially pure hydrogen is sought. In addition to the hydrogen-containing stream the hydrogen recovery stage also produces an offgas stream which comprises constituents inert under the conditions of the methanol synthesis and smaller amounts of unconverted carbon oxides.

The conversion of the synthesis gas stream and the residual gas stream to afford methanol (and water) is carried out over the methanol synthesis catalyst. The conversion is carried out in a synthesis loop, i.e. synthesis gas not converted over the catalyst is recycled as residual gas stream to the inlet of the relevant reactor and converted into methanol over the methanol synthesis catalyst together with synthesis gas used the first time. The conversion over the methanol synthesis catalyst is preferably carried out at a catalyst temperature of 180° C. to 270° C., or of 200° C. to 270° C., or of 220° C. to 270° C., and preferably a pressure of 55 bar to 100 bar. The conversion of the methanol synthesis catalyst is preferably carried out in one or more serially arranged or parallel reactor stages, wherein each of the reactor stages comprises an appropriate catalyst bed. The reactor stages especially comprise a water-cooled reactor and a gas-cooled reactor arranged downstream of the water-cooled reactor. Suitable catalysts are copper-based materials known from the prior art and comprising copper as the catalytically active species, one example thereof being a catalyst composition comprising copper, zinc oxide and aluminium oxide.

A preferred embodiment of the process according to the invention is characterized in that a portion of the synthesis gas stream is separated and combined with the purge gas stream to obtain a mixed synthesis gas stream and the mixed synthesis gas stream is sent to the hydrogen recovery stage for producing the hydrogen-containing stream.

If not enough purge gas is available for producing the hydrogen-containing stream, a portion of the synthesis gas stream may be separated and combined with the purge gas stream. This affords a mixed synthesis gas stream composed of synthesis gas stream and purge gas stream which is supplied to the hydrogen recovery stage.

A preferred embodiment of the process according to the invention is characterized in that the synthesis gas stream is compressed and a portion of the compressed synthesis gas stream is separated and combined with the purge gas stream. The synthesis gas stream is preferably compressed to synthesis pressure. The synthesis gas stream is preferably compressed to a pressure of not less than 55 bar and not more than 100 bar. It is preferable in this connection when the residual gas stream is compressed and combined with the compressed synthesis gas stream and the combined streams are passed through the bed of the methanol synthesis catalyst. The purge gas stream is especially diverted from the residual gas stream prior to the compression of the residual gas stream. The residual gas stream is preferably compressed to synthesis pressure. The residual gas stream is preferably compressed to a pressure of not less than 55 bar and not more than 100 bar.

A preferred embodiment of the process according to the invention is characterized in that the hydrogen-containing stream is compressed by a hydrogen compressor and the compressed hydrogen-containing stream is combined with the make-up gas stream to obtain the synthesis gas stream. In one embodiment the hydrogen-containing stream is compressed by the hydrogen compressor to a pressure which is about 1 to 2 bar above the pressure of the make-up gas. In one example the make-up gas has a pressure of 25 to 60 bar. It is preferable in this connection when the synthesis gas stream and the residual gas stream are compressed and passed through the bed of the methanol synthesis catalyst together. The synthesis gas stream and the residual gas stream are preferably compressed to synthesis pressure together. The synthesis gas stream and the residual gas stream are in particular compressed to a pressure of not less than 55 bar and not more than 100 bar together. The purge gas stream is thus diverted from the residual gas stream prior to the common compression of the residual gas stream and the synthesis gas stream.

A preferred embodiment of the process according to the invention is characterized in that the molar flow rate proportion of the synthesis gas stream in the mixed synthesis gas stream is between 0.10 and 0.95, preferably between 0.20 and 0.90, more preferably between 0.30 and 0.80 and more preferably between 0.50 and 0.75.

The molar flow rate may be reported for example in the units “kmol/h” (kilomol per hour).

A preferred embodiment of the process according to the invention is characterized in that the molar flow rate proportion of the portion separated from the synthesis gas stream based on the total molar flow rate of synthesis gas is between 0.001 and 0.999, preferably between 0.005 and 0.800, more preferably between 0.010 and 0.500, more preferably between 0.020 and 0.200 and more preferably between 0.050 and 0.100.

A preferred embodiment of the process according to the invention is characterized in that the synthesis gas stream has a stoichiometry number SN of 1.60 to 1.999, preferably of 1.80 to 1.999, more preferably of 1.85 to 1.999 and more preferably of 1.88 to 1.999.

A preferred embodiment of the process according to the invention is characterized in that the synthesis gas stream has a stoichiometry number SN of 1.85 to 1.95, preferably of 1.88 to 1.95.

In a further embodiment of the process according to the invention, the synthesis gas stream has a stoichiometry number SN of 1.60 to 1.95, or of 1.60 to 1.90, or of 1.80 to 1.95, or of 1.85 to 1.90.

A preferred embodiment of the process according to the invention is characterized in that the make-up gas stream has a stoichiometry number SN of less than 2.0, preferably of 1.60 to 1.95, more preferably of 1.70 to 1.90 and more preferably of 1.75 to 1.85. Synthesis gas from autothermal reforming often has a stoichiometry number of 1.80.

A preferred embodiment of the process according to the invention is characterized in that the hydrogen recovery stage comprises a pressure swing adsorption apparatus for separating hydrogen from the mixed synthesis gas stream. A pressure swing adsorption apparatus makes it possible to produce pure or at least virtually pure hydrogen at high pressures, for example at 40 to 60 bar. When hydrogen is already provided at high pressure by the hydrogen recovery stage, subsequent compressor stages, for example for compressing hydrogen (hydrogen compressor) or for compressing the synthesis gas stream, may be made correspondingly smaller. The concentration of inert constituents in the synthesis loop moreover increases ever slower the higher the purity of the hydrogen produced in the hydrogen recovery stage.

As an alternative to a pressure swing adsorption apparatus, the hydrogen recovery stage may also comprise a membrane separation stage for separating hydrogen from the mixed synthesis gas stream. Likewise conceivable are combinations of one or more pressure swing adsorption apparatuses and one or more membrane separation stages.

A preferred embodiment of the process according to the invention is characterized in that the hydrogen-containing stream has a hydrogen proportion of at least 80% by volume, preferably of at least 85% by volume, more preferably of at least 90% by volume, more preferably of at least 95% by volume, more preferably of at least 99% by volume, more preferably of at least 99.5% by volume and more preferably of at least 99.9% by volume.

The abovementioned objects are further at least partially achieved by a plant for producing methanol comprising the following plant components in fluid connection with one another: A reformer unit for producing a make-up gas stream comprising hydrogen and carbon oxides; a hydrogen recovery stage for producing a hydrogen-containing stream, wherein the reformer unit and the hydrogen recovery stage are configured such that a synthesis gas stream having a stoichiometry number SN, defined as SN=[n(H₂)−n(CO₂)]/[n(CO)+n(CO₂)], of less than 2.0 is obtainable from the hydrogen-containing stream and the make-up gas stream; a reactor stage comprising a methanol synthesis catalyst bed, wherein the reactor stage is configured such that the synthesis gas stream and a residual gas stream may be passed through the methanol synthesis catalyst bed at elevated pressure and elevated temperature, thus making it possible to obtain a product stream comprising methanol and the residual gas stream; a cooling apparatus for cooling the product stream, wherein the cooling apparatus is configured such that methanol may be separated from the residual gas stream and wherein the plant is configured such that a portion of the residual gas stream may be separated as a purge gas stream and the purge gas stream may be sent to the hydrogen recovery stage for producing the hydrogen-containing stream.

One embodiment of the plant according to the invention is characterized in that a portion of the synthesis gas stream may be separated and combined with the purge gas stream, thus making it possible to obtain a mixed synthesis gas stream, and the mixed synthesis gas stream may be sent to the hydrogen recovery stage for producing the hydrogen-containing stream.

That is, according to the aforementioned embodiment, the plant according to the invention is configured such that a portion of the synthesis gas stream may be separated and combined with the purge gas stream, thus making it possible to obtain a mixed synthesis gas stream, and the mixed synthesis gas stream may be sent to the hydrogen recovery stage for producing the hydrogen-containing stream.

In one embodiment the plant comprises a plurality of reactor stages. In particular each of the plurality of reactor stages comprises a methanol synthesis catalyst bed.

In one embodiment the reactor stages of the plurality of reactor stages are arranged in series.

In a further embodiment the reactor stages of the plurality of reactor stages are arranged in parallel.

In a further embodiment each reactor stage of the plurality of reactor stages has a cooling apparatus for cooling the product stream arranged downstream of it, wherein the cooling apparatus is configured such that methanol may be separated from the residual gas stream.

The aforementioned objects are further at least partially achieved by the use of the process according to the invention or of the plant according to the invention for producing methanol from make-up gas produced by autothermal reforming and/or partial oxidation and/or combined reforming and/or pyrolysis of biomass.

“Combined reforming” is understood by those skilled in the art to mean a combination of autothermal reforming (ATR) and steam reforming (SMR).

### BRIEF DESCRIPTION OF THE DRAWINGS

The invention is more particularly elucidated hereinbelow by way of three inventive examples as well as one comparative example and one numerical example without in any way limiting the subject-matter of the invention. Further features, advantages and possible applications of the invention will be apparent from the Substitute Specification following description of the working examples in connection with the drawings and the numerical examples.

In the figures

FIG. 1 shows a schematic block flow diagram of a production process or a plant 100 for methanol synthesis according to a first exemplary embodiment of the invention,

FIG. 2 shows a schematic block flow diagram of a production process or a plant 200 for methanol synthesis according to a second exemplary embodiment of the invention,

FIG. 3 shows a schematic block flow diagram of a production process or a plant 300 for methanol synthesis according to a third exemplary embodiment of the invention,

FIG. 4 shows a schematic block flow diagram of a production process or a plant 400 for methanol synthesis according to the prior art.

### DETAILED DESCRIPTION OF THE PREFERRED EMBODIMENTS

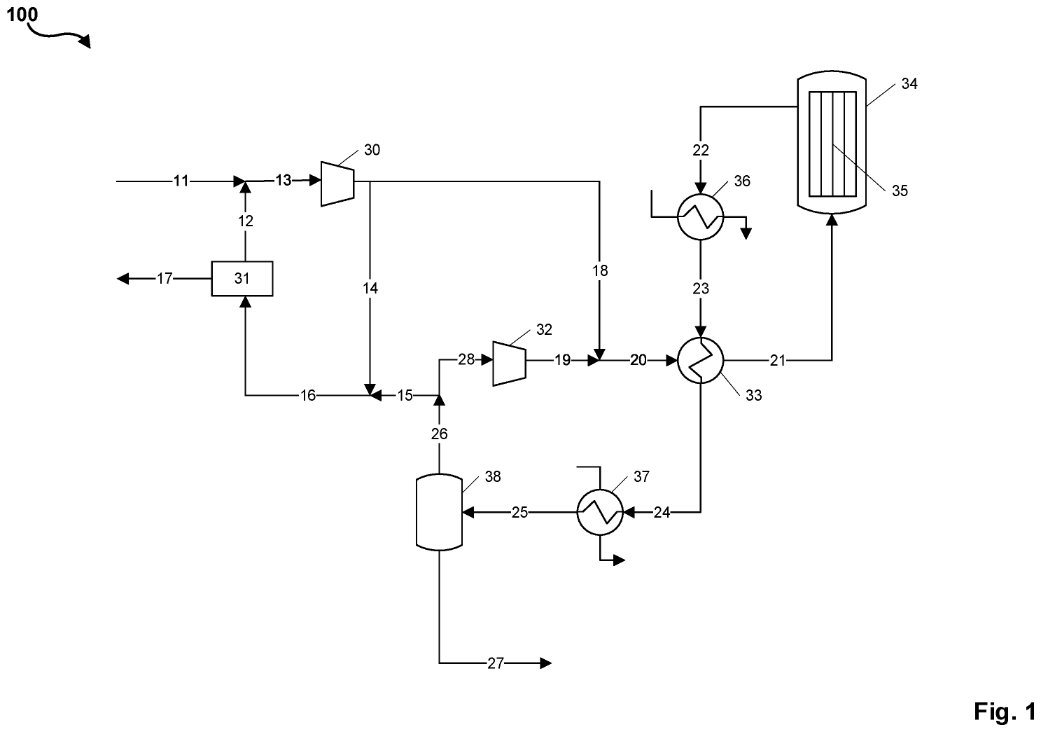

In the process mode according to FIG. 1 a make-up gas stream 11, for example produced in a plant for autothermal reforming of natural gas (not shown), is combined with a hydrogen-containing stream 12 to produce a synthesis gas stream 13 having a stoichiometry number of less than 2.0. The synthesis gas stream 13 is thus substoichiometric having regard to the hydrogen demand of the methanol synthesis. The synthesis gas stream 13 is compressed to synthesis pressure by a compressor stage 30. A portion of the synthesis gas stream 13 is separated as synthesis gas substream 14 and combined with a purge gas stream 15 to afford a mixed synthesis gas stream 16. The mixed synthesis gas stream 16, which comprises the purge gas stream 15 and the separated synthesis gas stream 14 (synthesis gas substream), is sent to the hydrogen recovery stage 31, in which by pressure swing adsorption the hydrogen-containing stream 12 is produced with a hydrogen proportion of at least 99% by volume. In an equivalent alternative of the process shown the purge gas stream 16 and the synthesis gas substream 14 may also be supplied to the hydrogen recovery stage 31 as separate streams. Offgas 17 simultaneously produced in the hydrogen recovery stage 31 and containing carbon oxides and constituents inert under the conditions of the methanol synthesis may be used for example as a fuel gas in the reformer unit arranged upstream of the methanol synthesis.

The main portion 18 of the synthesis gas stream compressed to synthesis pressure is combined with a residual gas stream 19 compressed to synthesis pressure in a compressor stage 32. The resulting combined synthesis gas stream 20 is heated in a heat exchanger 33 and as heated combined synthesis gas stream 21 sent to a water-cooled methanol reactor 34. The methanol reactor 34 carries out the conversion of the synthesis gas from combined synthesis gas stream 21 over the methanol synthesis catalyst of the catalyst bed 35 to afford methanol and water. The product stream 22 resulting from the conversion in the reactor 34 which comprises not only methanol and water but also unreacted synthesis gas or residual gas is then consecutively cooled via the heat exchangers 36, 33 and 37, the product streams 23, 24 and 25 resulting downstream of the respective heat exchangers. A separator 38 subsequently carries out the separation of the cooled product stream 25 into a liquid phase comprising methanol and water and a gaseous phase comprising residual gas. The synthesis gas not converted in reactor 34, i.e. residual gas, is withdrawn from the separator 38 as residual gas stream 26. A crude methanol stream 27 comprising methanol and water is simultaneously withdrawn from the separator 38 and sent for further workup, for example a rectification (not shown). The purge gas stream 15 is separated from the residual gas stream 26 and a remaining residual gas stream 28 is compressed to synthesis pressure in the compressor stage 32. Residual gas stream 19 compressed to synthesis pressure is in turn combined with synthesis gas stream 18 and returned to the conversion to afford methanol in the methanol reactor 34.

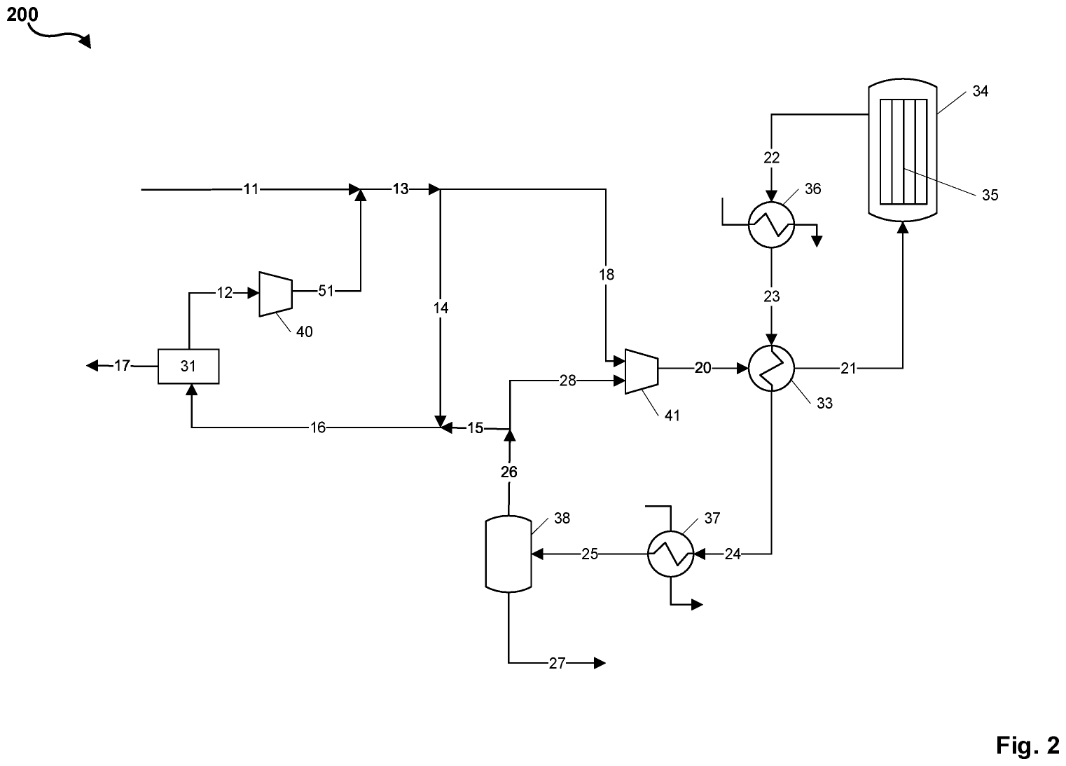

FIG. 2 shows a type of process mode according to a further inventive example which is modified compared to the example of FIG. 1. In the process mode according to FIG. 2 the hydrogen-containing stream 12 produced in the hydrogen recovery stage 31 is compressed in a hydrogen compressor 40 to obtain a compressed hydrogen-containing stream 51 which is combined with the make-up gas stream 11. This affords a synthesis gas stream 13 of which the main portion 18 is sent to compressor stage 41 for compression to synthesis pressure and of which a portion is diverted as synthesis gas substream 14 and combined with the purge gas stream 15. The mixed synthesis gas stream 16 results from the streams 14 and 15. The synthesis gas stream 18 and the residual gas stream are together sent to a compressor stage 41. Compressor stage 41 has two ports on its suction side which allows simultaneous compression of the synthesis gas stream 18 and the residual gas stream 28 to obtain the combined synthesis gas stream 20 which is heated in heat exchanger 33 and sent as combined synthesis gas stream 21 to the methanol reactor 34.

That which is recited in connection with FIG. 1 applies correspondingly to the further elements shown in FIG. 2.

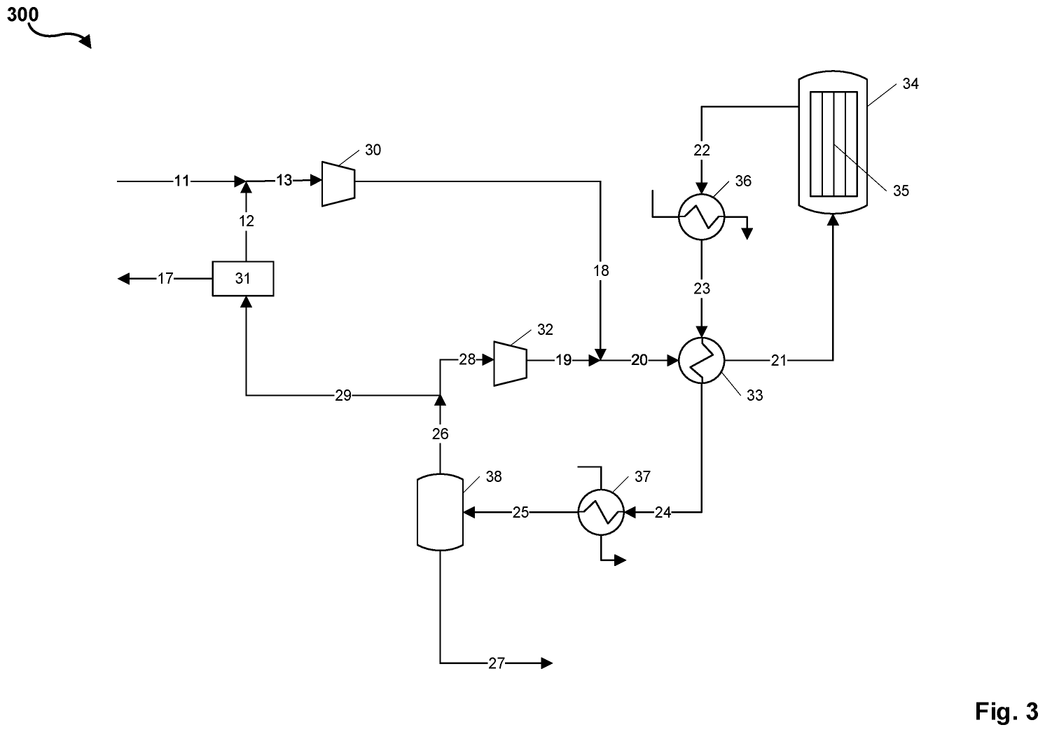

FIG. 3 shows a process mode which is modified compared to the example of FIG. 1. According to the flow scheme of FIG. 3 no synthesis gas substream that is combined with a purge gas stream is separated from the synthesis gas stream 18. On the contrary, the residual gas stream 26 has a portion withdrawn from it as a purge gas stream and exclusively this portion withdrawn from the residual gas stream 26 is supplied to the hydrogen recovery stage 31 as purge gas stream 29.

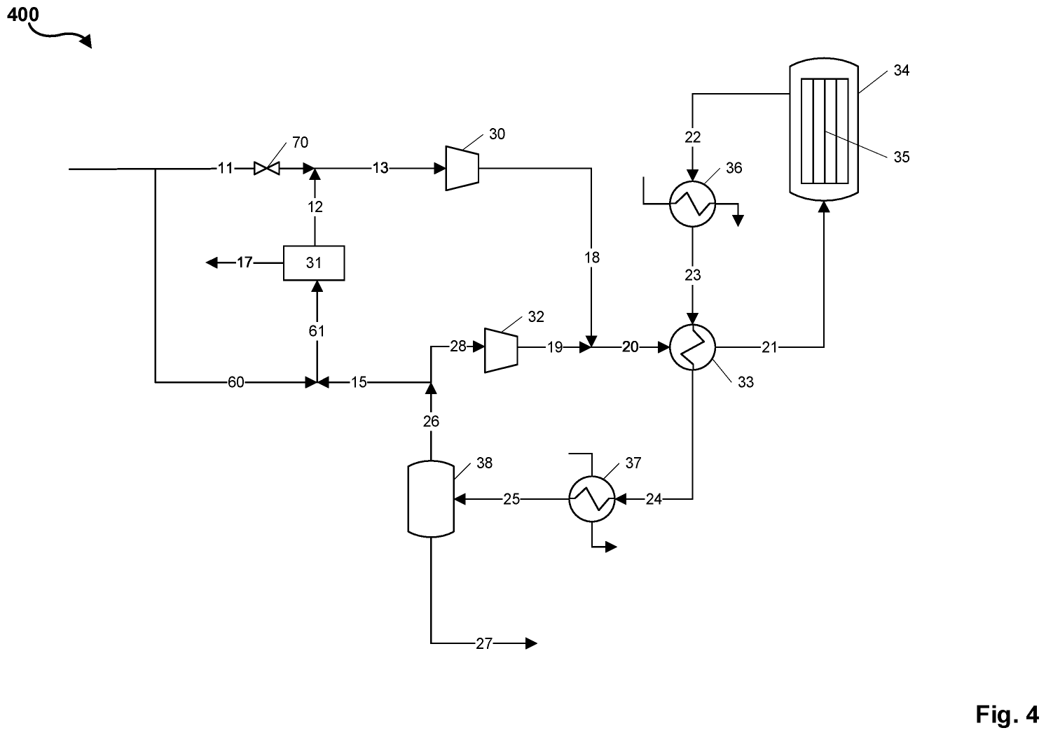

FIG. 4 shows a type of process mode known from the prior art. Here too, a mixed gas stream of synthesis gas and purge gas is sent to the hydrogen recovery stage 31 and utilized for hydrogen recovery. However, the synthesis gas proportion of the mixed gas stream is a make-up gas substream 60 which is diverted from the (main) make-up gas stream 11 using the throttle means 70. The make-up gas substream 60 and the purge gas stream 15 are combined and as mixed synthesis gas stream 61 sent to the hydrogen recovery stage 31. In contrast to the above inventive examples, the mixed synthesis gas stream 61 is thus not produced from synthesis gas already enriched with hydrogen and purge gas but rather from make-up gas and purge gas.

The following tabulated numerical example illustrates the technical advantage of the process according to the invention and of the plant according to the invention having regard to the use of synthesis gas having a stoichiometry number of less than 2.0, in particular having regard to the process of post-published application EP 19020610.2. The data shown are based on a simulation corresponding to the process mode of FIG. 1. It may be possible to omit the separation of a synthesis gas substream 14 depending on the need for hydrogen from hydrogen recovery stage 31 which especially in Examples 1 to 3 is correspondingly also low on account of the low stoichiometry number. The process mode then corresponds to that of FIG. 3.

The simulation was performed using Aspen Plus® software. Six Inventive Examples 1 to 6 (synthesis gas stoichiometry number of less than 2.0) and two Comparative Examples 1 and 2 (stoichiometry number of 2.0 or more) are shown.

Synthesis in a water-cooled methanol reactor is carried out at a synthesis pressure of 80 bar and a reactor outlet temperature of 235° C. at a production of 5000 tonnes per day of methanol.

The recirculation rate R, defined as

$$R = \frac{V olume flow ( residual gas stream )}{V olume flow ( synthesis gas stream )} ,$$

is 2.5 in all examples. In other words, the volume flow of the recycled residual gas stream 19 is 2.5 times the volume flow of the synthesis gas stream 18. The stoichiometry number of the combined synthesis gas stream 20/21 at the inlet of the water-cooled methanol reactor before conversion into methanol is derived from the stoichiometry numbers of the synthesis gas stream 18, the residual gas stream 20 and the recirculation rate. The recirculation rate is typically adjusted such that a total carbon conversion of at least 80%, preferably of at least 85% and more preferably of at least 95% is achieved. Recirculation rates in a range from 1.5 to 4.5 are typical.

The columns of the following table show from left to right
the stoichiometry number SN of the synthesis gas stream 18;
the stoichiometry number SN of the combined synthesis gas stream 20 resulting from synthesis gas stream 18 and residual gas stream 19;
the natural gas flow required for production of the make-up gas 11 (as mass flow in kg/hr) which is supplied to the reformer unit upstream of the methanol synthesis;
the conversion of hydrogen (H₂) in the synthesis loop;
the conversion of carbon monoxide (CO) in the synthesis loop;
the conversion of carbon dioxide (CO₂) in the synthesis loop and
the hydrogen stream withdrawn from the hydrogen recovery stream 31 (as molar flow rate in kmol/hr).

| | | | | | | | Hydrogen |
|---|---|---|---|---|---|---|---|
| | | | Natural | | Carbon | Carbon | flow from |
| | SN | SN | gas | Hydrogen | monoxide | dioxide | recovery |
| | (synthesis | (reactor | flow/ | conversion | conversion | conversion | stage/ |
| | gas) | inlet) | (kg/hr) | in % | in % | in % | (kmol/hr) |
| Example 1 | 1.884 | 1.906 | 119974 | 97.1 | 99.1 | 74.6 | 741 |
| Example 2 | 1.900 | 2.108 | 120310 | 97.2 | 99.2 | 76.6 | 834 |
| Example 3 | 1.926 | 2.503 | 120866 | 97.1 | 99.4 | 79.8 | 990 |
| Example 4 | 1.953 | 3.006 | 121455 | 97.0 | 99.5 | 83.0 | 1158 |
| Example 5 | 1.977 | 3.507 | 121995 | 96.8 | 99.6 | 85.5 | 1308 |
| Example 6 | 1.999 | 4.009 | 122480 | 96.4 | 99.7 | 87.4 | 1452 |
| Comparative | 2.019 | 4.500 | 122929 | 96.1 | 99.7 | 88.9 | 1591 |
| Example 1 | | | | | | | |
| Comparative | 2.041 | 5.001 | 123413 | 95.5 | 99.7 | 90.2 | 1748 |
| Example 2 | | | | | | | |

In Examples 1 to 6 (inventive) the stoichiometry number of the synthesis gas 18 is between 1.884 and 1.999, thus below 2.0 in every case. In Comparative Examples 1 and 2 the stoichiometry number of the synthesis gas 18 is 2.019 and 2.041, thus not less than 2.0 in every case. The stoichiometry number of the combined synthesis gas 20/21 supplied at the reactor inlet of the water-cooled methanol reactor 34 increases continuously from 1.906 to 4.009 (Examples 1 to 6) and 4.500 and 5.001 (Comparative Examples 1 and 2) with increasing stoichiometry number of the synthesis gas 18 at a constant recirculation rate of 2.5.

The mass flow of natural gas required for methanol production decreases continuously with decreasing stoichiometry number of the synthesis gas 18. Thus for example a saving of 2955 kg/hr, corresponding to a saving of 2.4%, is achieved between Comparative Example 1 (stoichiometry number 2.019) and Example 1 (stoichiometry number 1.906) The process according to the invention exhibits the further advantage that the conversions of hydrogen are particularly high at a low stoichiometry number of the synthesis gas 18 according to Examples 1 to 6. It is apparent that the hydrogen conversion continuously increases to above 97% with decreasing stoichiometry number of the synthesis gas 18.

The conversion of carbon monoxide is virtually constant over the entire stoichiometry number range of Examples 1 to 6 and Comparative Examples 1 and 2.

The process according to the invention further exhibits the advantage that the conversions of carbon dioxide, i.e. of methanol formed from carbon dioxide, proportionally decrease with decreasing stoichiometry number. Accordingly the carbon dioxide conversion is only 74.6% in Example 1 but already 88.9% in Comparative Example 1 and already above 90% in Comparative Example 2. This has the advantage that less water requiring subsequent separation is formed in the synthesis loop and also that more purge gas for supplying to the hydrogen recovery stage is available. The latter advantage means that there is no longer any need potentially to use larger amounts of reformed gas (synthesis gas substream 14) to produce the hydrogen which would then no longer be available for conversion in the synthesis loop.

It is thus further possible to cover the hydrogen demand generated by the hydrogen recovery stage exclusively by supplying purge gas to the hydrogen recovery stage. Under appropriate conditions it is then unnecessary to divert a synthesis gas substream 14 from the synthesis gas stream 18, as shown in FIG. 3.

The process according to the invention and a corresponding plant generally have the further advantage that provision of a synthesis gas having a low stoichiometry number requires provision of less hydrogen from a hydrogen recovery stage. The hydrogen recovery stage may thus be made smaller, thus resulting in a reduction of the capital costs (CAPEX) for the relevant plant.

### LIST OF REFERENCE SYMBOLS

100, 200, 300 Process, plant (invention)
400 Process, plant (prior art)
11 Make-up gas stream
12, 51 Hydrogen-containing stream
13, 18 Synthesis gas stream
14 Synthesis gas substream
15, 29 Purge gas stream
16 Mixed synthesis gas stream (invention)
17 Offgas
19, 26, 28 Residual gas stream
20, 21 Combined synthesis gas stream
22, 23, 24, 25 Product stream
26 Residual gas stream
27 Crude methanol stream
30, 32, 41 Compressor stage
31 Hydrogen recovery stage
34 Water-cooled methanol reactor
35 Catalyst bed
33, 36, 37 Heat exchanger
38 Separator
40 Hydrogen compressor
60 Make-up gas substream
61 Mixed synthesis gas stream (prior art)
70 Throttle means

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1.-17. (canceled)

18. A process for producing methanol, comprising:
admixing a make-up gas stream from a reformer unit comprising hydrogen and carbon oxides with a hydrogen-containing stream from a hydrogen recovery stage to obtain a synthesis gas stream having a stoichiometry number SN, defined as SN=[n(H₂)−n(CO₂)]/[n(CO)+n(CO₂)], of less than 2.0,
combining the synthesis gas stream with a residual gas stream and the synthesis gas stream and the residual gas stream are passed through a bed of a methanol synthesis catalyst at elevated pressure and elevated temperature to obtain a product stream comprising methanol and the residual gas stream,
cooling the product stream is cooled to separate methanol from the residual gas stream, and
separating a portion of the residual gas stream as a purge gas stream and sending the separated portion to the hydrogen recovery stage for producing the hydrogen-containing stream.

19. The process according to claim 18, wherein a portion of the synthesis gas stream is separated and combined with the purge gas stream to obtain a mixed synthesis gas stream and the mixed synthesis gas stream is sent to the hydrogen recovery stage for producing the hydrogen-containing stream.

20. The process according to claim 18, wherein the synthesis gas stream is compressed and a portion of the compressed synthesis gas stream is separated and combined with the purge gas stream.

21. The process according to claim 20, wherein the residual gas stream is compressed and combined with the compressed synthesis gas stream and the combined streams are passed through the bed of the methanol synthesis catalyst.

22. The process according to claim 18, wherein the hydrogen-containing stream is compressed by a hydrogen compressor and the compressed hydrogen-containing stream is combined with the make-up gas stream to obtain the synthesis gas stream.

23. The process according to claim 22, wherein the synthesis gas stream and the residual gas stream are compressed and passed through the bed of the methanol synthesis catalyst together.

24. The process according to claim 19, wherein the molar flow rate proportion of the synthesis gas stream in the mixed synthesis gas stream is between 0.10 and 0.95.

25. The process according to claim 19, wherein the molar flow rate proportion of the portion separated from the synthesis gas stream based on the total molar flow rate of synthesis gas is between 0.001 and 0.999.

26. The process according to claim 18, wherein the synthesis gas stream has a stoichiometry number SN of 1.60 to 1.999.

27. The process according to claim 18, wherein the synthesis gas stream has a stoichiometry number SN of 1.85 to 1.95.

28. The process according to claim 18, wherein the make-up gas stream has a stoichiometry number SN of less than 2.0.

29. The process according to claim 18, wherein the hydrogen recovery stage comprises a pressure swing adsorption apparatus for separating hydrogen from the mixed synthesis gas stream.

30. The process according to claim 18, wherein the hydrogen recovery stage comprises a membrane separation stage for separating hydrogen from the mixed synthesis gas stream.

31. The process according to claim 18, wherein the hydrogen-containing stream has a hydrogen proportion of at least 80% by volume.

32. A plant for producing methanol comprising the following plant components arranged in fluid connection with one another:
a reformer unit for producing a make-up gas stream comprising hydrogen and carbon oxides;
a hydrogen recovery stage for producing a hydrogen-containing stream, wherein the reformer unit and the hydrogen recovery stage are configured such that a synthesis gas stream having a stoichiometry number SN, defined as SN=[n(H₂)−n(CO₂)]/[n(CO)+n(CO₂)], of less than 2.0 is obtainable from the hydrogen-containing stream and the make-up gas stream;
a reactor stage comprising a methanol synthesis catalyst bed, wherein the reactor stage is configured such that the synthesis gas stream and a residual gas stream may be passed through the methanol synthesis catalyst bed at elevated pressure and elevated temperature,
thus, making it possible to obtain a product stream comprising methanol and the residual gas stream;
a cooling apparatus for cooling the product stream, wherein the cooling apparatus is configured such that methanol may be separated from the residual gas stream; and wherein
the plant is configured such that a portion of the residual gas stream may be separated as a purge gas stream and the purge gas stream may be sent to the hydrogen recovery stage for producing the hydrogen-containing stream.

33. The plant according to claim 32, wherein a portion of the synthesis gas stream may be separated and combined with the purge gas stream, thus making it possible to obtain a mixed synthesis gas stream, and the mixed synthesis gas stream may be sent to the hydrogen recovery stage for producing the hydrogen-containing stream.
